(19) United States
(12) Patent Application Publication
(10) Pub. No.: US 2018/0132751 A1
(43) Pub. Date: May 17, 2018

(54) AUTOMATIC SYSTEM AND METHOD FOR EVALUATING AND TREATING VISUAL HEALTH

(71) Applicant: Medisim, LTD., Neve Ilan (IL)

(72) Inventor: Moshe Yarden, Neve Ilan (IL)

(21) Appl. No.: 15/812,153

(22) Filed: Nov. 14, 2017

Related U.S. Application Data
(60) Provisional application No. 62/421,587, filed on Nov. 14, 2016.

(51) Int. Cl. A61B 5/0484; A61B 5/0478; A61B 5/00; A61B 5/0482; A61B 3/10

(52) U.S. Cl. CPC A61B 5/04842; A61B 5/0478; A61B 5/7445; A61B 3/10; A61B 5/0482; A61B 5/4809; A61B 5/4836; A61B 5/6803

(57) ABSTRACT

A system and method is useful for treating amblyopia and other conditions by visual stimulation of the brain. Automatic discrimination between a pair or more of similar body organs, such as the eyes is accomplished for improved safety and efficacy of a treatment. Automatic measurement of a physiological parameter or state in response to a stimulation treatment (responsiveness) over time is also accomplished. Measurements are done over time, within the same treatment session, or between the same treatment session to allow for adapting the visual stimulation used for test and assessment purposes, visual stimulation used for treatment, or a combination of testing and treatment.

### CROSS-REFERENCE TO RELATED APPLICATIONS

The present application claims the benefit of the filing date of U.S. Provisional Patent Application No. 62/421,587 filed Nov. 14, 2016, the disclosure of which are hereby incorporated herein by reference.

### FIELD OF USE

This disclosure relates to a system and method useful for treating, among other things, amblyopia by stimulation of the brain. In particular, the present disclosure relates to a system and method of treating amblyopia by optimizing the treatment based on responsiveness over time to a treatment.

### BACKGROUND

Visual and auditory stimulation is known as a way to stimulate the brain. Stimulation therapies intend to strengthen synaptic connections in the relevant brain region. The eyes and ears are connected to the nervous system and endocrine system. For example, the eyes which through the light receptors of the retinas send electrical messages to the visual cortex and also to the hypothalamus. The light coming in through the eyes can augment the function of the nervous system and the endocrine system. Therapy modalities, may range from right light therapy, flickering or oscillations of characteristics of images. Each therapy has its effect on various regions or functions of the brain.

Treatment of amblyopia using repetitive visual stimulation enhances recovery from severe amblyopia. Whereas, visual stimulation will rapidly enhance visually evoked responses to novel stimuli and enhance the recovery from severe amblyopia (Montey K L, Eaton N C, Quinlan E M, Learn Mem Journal 2013). Amblyopia is generally defined by the America Association for Pediatric Ophthalmology and Strabismus (AAPOS) as decreased vision in one or both eyes due to abnormal development of vision in infancy or childhood. Vision loss occurs because nerve pathways between the brain and eye are not properly stimulated. The brain “learns” to see only blurry images with the amblyopic eye even when glasses are worn. As a result, the brain favors one eye, usually due to poor vision in the other eye. Amblyopia is the leading cause of vision loss amongst children.

Another example is the case of Amblyaudia, a deficit in binaural integration of environmental information entering the auditory system. In Amblyaudia, a suppression of activity occurs in the non-dominant auditory pathway (ear) by activity in the dominant pathway, much like in Amblyopia. Computer-based auditory training programs are targeting treatment of such Auditory Processing Disorders, by way of providing various stimulations. Objective measurement of a physiological parameter or state, prior, during or after applying a treatment, and more specifically after applying stimulation based therapy, is instrumental for optimal and correct treatment.

There are various techniques used by medical professionals to measure sensory activity in a patient. Visual Evoked Potential (VEP) measures the electrical activity in the vision system. When light from an image enters the eye, it is converted into electrical energy at the retina and travels through the optic nerve to the visual cortex of the brain which processes vision. Auditory Evoked Potentials (AEP) is very small auditory evoked potentials in response to an auditory stimulus, which are recorded by electrodes placed on the scalp.

State of eye care at the present time primarily relies on in-office subjective evaluation of the patient. Even when VEP is used, the complexity and required know-how by the care giver to make use of objective measurements and data is challenging even for the medical professionals. For this reason, the use of VEP is limited to being an ancillary and observant input rather than a parameter driven tool for setting an optimal treatment modality. VEP is a very important tool in understanding the complex amblyopic mechanism. However, the above drawbacks of complexity and interpretative results leave this tool less desirable. Current techniques for treating conditions like patching the unaffected eye for amblyopia have been met with various disadvantages. Professionals have noted that special attention should be paid to amblyopic treatment as patching can have a negative effect on the sound eye. (Regina Halfeld Furtado de Mendonça, Int Ophthalmol. 2013; 33(5): 515-519).

Thus there still remains a strong felt and unmet need to provide a system that seamlessly measures parameters of a condition and provides a treatment regime based on such measurements.

### SUMMARY

The present inventions solve the drawbacks of current state of the art devices and systems and meet the above current needs as well as giving additional benefits. For purposes of describing the present invention as principals are similar, references made to VEP and the visual system and pathway are also applicable to AEP auditory system and pathway, and vice versa.

It is an object of the present invention to provide a system and method useful for treating, among other things, amblyopia by stimulation of the brain. Stimulation may be accomplished visually, auditory, sensory of taste, touch, smell, or any other type of stimulation to the brain, and any combination thereof.

It is also an object to automatically discriminate between a pair (or more) of similar body organs, such as the eyes. Such a process is advantageous as it facilitates for improved safety and efficacy of a treatment. Identifying the correct organ to treat will help assure that it is actually the one being treated. At the same time, such discriminating may safeguard the fellow organ from being mistakenly treated.

Another object is to automatically measure a physiological parameter or state in response to a stimulation treatment (responsiveness) over time. This process is advantageous as it establishes the efficacy of the delivered treatment. Measurements done over time, within same treatment session, or between same treatment session, allows for adapting the visual stimulation used for test and assessment purposes, visual stimulation used for treatment, or a combination thereof.

Yet another object is to automatically determine a change in a treatment parameter in connection with a measured responsiveness over time. This object is desirable as it allows for optimizing the treatment based on responsiveness. The ability to automatically re-measure responsiveness over time to a treatment is another object and upon a need to change a treatment's parameters. This process is advantageous because it adjusts the delivered treatment in relationship to the progress or lack of treatment's efficacy. For example, the determination criteria may be with respect to threshold values, or trends noticed in the responsiveness. The ability to automatically re-measure responsiveness over time to a treatment and to present this information to the patient and or caretaker is useful and another object of the invention for providing the use with feedback re-progress of the treatment and by this to support compliance. This ability is also useful for optimizing treatment period and setting periodical in-office checkups. The ability to automatically detect the state of awakens of a user is advantageous as it allows to provide indication to a caretaker (or a user) that the person using the device is asleep, his eyes are shut, and thus optimal visual stimuli is not delivered directly to the eyes.

In the case of treating a visual disorder, the disclosed invention, is useful for the: (a) measurement of a physiological parameter or state, prior, during or after applying a treatment, (b) automatically discriminate between the states of health of a patient's eyes, (c) automatic measurement of the responsiveness over time to a treatment (d) automatic determining a due change in a treatment's parameter, i.e. stimulation, and (e) initiating a feedback to the patient or caretaker, or (f) optimizing parameters of treatment and (h) identifying and alerting of the state of awakeness of the user.

### BRIEF DESCRIPTION OF THE DRAWINGS

FIG. 1 illustrates a head mounted mechanical arrangement and an electrode for measuring a signal formed in response to visual stimulation.

FIG. 2 illustrates automatic evaluation of responsiveness to a visual stimulation based treatment, and adjusting the stimulation parameters in real-time.

FIG. 3 illustrates evaluation of responsiveness to a stimulation based treatment, and the same stimulation used to test responsiveness, treat a patient or both treat and test including informing a caretaker of a need to adjust parameters.

FIG. 4 illustrates automatic discrimination between two eyes of a patient to identify the eye with lesser health and in need for treatment and depicts the same stimuli may be used either for test purposes or for treatment purposes.

FIG. 5 illustrates automatic evaluation of responsiveness to a visual stimulation based treatment using therapeutic stimuli and adjusting the stimulation parameters.

FIG. 6 illustrates automatic evaluation of responsiveness to a visual stimulation based treatment using dedicated test stimuli and adjusting the stimulation parameters.

FIG. 7 illustrates automatic evaluation of responsiveness to a visual stimulation based treatment adjusting the stimulation parameters over time based on time elapsed re-evaluation of responsiveness.

### DETAILED DESCRIPTION

The following description is provided so as to enable any person skilled in the art to make use of the invention and sets forth the embodiments contemplated of carrying out this invention. Various modifications, however, will remain apparent to those skilled in the art, since the generic principles of the present invention have been defined specifically to provide product and method of the invention described herein.

The invention relates to combined therapeutic and diagnostic device, which involves detection of brain electric response to presentation of visual or other stimulation. More specifically, the present invention discloses a system for visual stimulation comprising means for objectively measuring brain and visual pathway responsiveness to a stimulation over time, and allowing for adaptation of the stimulation's parameters, according to the measured brain responsiveness and other considerations.

Depending on the embodiment, the disclosed system may include a closed-loop process, having a control-loop, by which the state of the patient's eye or eyes are assessed, and the assessment derived information is used to determine an adjustment of a stimuli used for treatment, re-assessment, or a combination thereof. The adjustment may be done in real-time, or at an otherwise determined point of time.

The adaptable stimulation parameters may include, for example, and are not limited to: (a) at least one image characteristic such as brightness, contrast, or any other conventional image parameters and their combinations, and/or (b) superimposed images and/or (c) static or moving objects and any combination thereof. The adaptation of any stimulation parameter characteristics or timing, in the present invention, for test purposes, treatment purposes, or both, may be done automatically by the disclosed system. Alternatively, adaptation may be made by a caretaker or other user, once the system provides an indication of a need to adapt said stimulation.

Stimulation responsiveness measurement is useful in order to assess electrical signal transient in the visual pathway and the brain. The responsiveness, for example but not limited to, an electrical signal transient in the visual pathway and the brain, is indicative of a physiological change in the visual pathway and the brain. Such change may be indicative of an efficacy of a stimulation based treatment applied to the visual pathway and the brain.

Furthermore, the stimulation responsiveness measurement of the present invention may be, depending on the embodiment, achieved using Visual Evoked Potential (VEP) sampling, recording and analyzing. VEP measures the electrical activity in the vision system. The principal of VEP is that as light from an image enters a subject's eye, it is transformed into electrical energy at the retina and travels through the optic nerve to the visual cortex of the brain which processes the received image. VEP test measures the Amplitude (strength) of the signal reaching the visual cortex and the Latency of the signal (how fast it gets there). An alternative stimulation responsiveness measurement of the present invention may be using Electroencephalography (EEG). EEG is an electrophysiological monitoring method to record electrical activity of the brain. EEG measures voltage fluctuations resulting from ionic current within the neurons of the brain. EEG refers to the recording of the brain's spontaneous electrical activity over a period of time, as recorded from electrodes in contact with the scalp. The stimulation responsiveness measurement may be utilized by one or more EEG electrode. The electrodes are disposed in at least one location out of a list comprising: the forehead area of the user, the top of the head, the ear or ears, behind the ear or ears, the back of the head or a combination of two or more sites. The electrode or electrodes disposed may operate separately or in conjunction with one another, or with the stimulation used for responses evaluation. The electrodes may, depending on the embodiment, be integrated into the mechanical arranging of the disclosed device, by way of non-limiting examples, the earphone housing or side arms at the side of the head, a strap or other fixing mean in contact with the top of the head or the back of the head, or the housing of the display components at the front of the head.

The stimulation responsiveness measurement is also useful for autonomous discrimination between a patient's two eyes. The discrimination is based on the different health state of each eye (and\or its visual pathway and respective brain area), as the difference in said health state is reflecting on responsiveness to stimulation. The present invention is useful for determining the health state of each eye with respect to the fellow eye but also to itself by comparing responsiveness measurements to a visual stimulation to each eye. The comparison between one eye responsiveness to the fellow eye is useful for determining which is a lesser healthy eye, for example which of the patient's eye is the Amblyopic eye.

The present invention also provides for comparison between same eye responsiveness over time and is useful for assessing progression of a medical condition, or recovery from a medical condition. The disclosed system of the present invention, includes, depending on the embodiment: (a) at least one display for delivering a visual stimulation, (b) a controller unit for controlling parameters of the stimulation coupled to the said display (c) at least one electrode in contact with the patient's sculpt, coupled to said controller unit for sensing an electrical signal, and; (d) a mechanical arrangement for affixing at least said display and electrode on a patient's head.

Depending on the embodiment the disclosed system may include, but not necessarily, components configured for automatic: (a) assessment of the measured responsiveness, (b) comparing time elapsed measurements of responsiveness, (c) determination of which eye is of lesser health, (d) determination the level of change in responsiveness over time; (e) setting the parameters of the visual stimulation to each or both eyes; or any combination thereof. The system may include a display for displaying information to the caretaker or user such as: treated eye, treatment progress, treatment parameters and the like.

Depending on the embodiment, the disclosed system may have two different sets of stimuli parameters: one for treatment and one for responsiveness measurement. In an embodiment of the present invention, the disclosed system is using the stimuli parameters used for treatment for responsiveness measurement. The system may configured to have an image processing capabilities for characterizing an image parameters presented to a patients eye and to link them to said responsiveness measured simultaneously. Further the disclosed system may be configured, but not necessarily, to change the visual stimulation parameters used for the assessment of responsiveness and/or for the treatment.

The type of stimuli of the stimulation used for the assessment or treatment may be changed in either real-time or not real-time, by way of non-limiting examples: (a)change form a static stimuli to a dynamic stimuli or vice versa, (b) from static image to a dynamic image or vice versa, (c) from a still object to a moving one or vice versa, (d) change of image parameters (brightness, contrast, hue, saturation, resolution etc.); whereas the change or changes are characterized by a duration an amplitude an interval, or any combination thereof. In an embodiment of the present invention, the disclosed system is configured to present same image, at different resolution to each eye. Depending on the embodiment, the disclosed system is configured to present same image, at different frame per second rate to each eye. The disclosed system may also be configured to set the frame per second rate and\or the resolution, for each eye separately, to both eyes or to each eye separately, in accordance to a certain ratio (predetermined or set in real-time). The frame rate and resolution may be set independently or dependently with respect to one another, or to another parameter of the disclosed system, or the user. In an embodiment of the present invention, a change of the visual stimulation used for the assessment of responsiveness or for the treatment, may be done by a caretaker.

In an embodiment of the present invention, a change the visual stimulation used for the assessment of responsiveness or for the treatment may be done autonomously by the system, being a learning system. The data collected form at least one assessment may be used for the system to determine a desired change in the visual stimulation. In an embodiment of the present invention, adjusting of the stimulation used for assessment of responsiveness or for the treatment may be performed at any points of time: (a) prior to a treatment session, (b) during a treatment session in real time or (c) at the end or immediately after a treatment session. Assessment of responsiveness over time many be carried with respect to either (a) within the same eye, comparing state of same eye over time, (b) comparing the state of the two eyes with respect to one another; or a combination thereof. The assessed responsiveness information, may be stored and analyzed for: (a) determining the state of the visual system at any given time for adjusting a treatment stimuli or an assessment stimuli during the treatment in real time or after the treatment off line, (b) for assessing changes in the state of the visual system over time, for example to identify effect of a treatment, and (c) for providing an overview of the changes to a care taker concerning a patient's visual system over time so to indicate efficacy of a treatment, (d) for providing a feedback to the patient relating to either the state of his visual system, his compliance with a treatment (administered by the disclosed system, or otherwise) or both, and (e) for demining a change in the parameters of the stimulation used as treatment.

In an embodiment of the present invention, an assessment of responsiveness may be performed at any point of time: (a) prior to a treatment session, (b) during a treatment session or (c) at the end or immediately after a treatment session. In an embodiment of the present invention, adjusting of the stimulation used for treatment may be performed at any point of time: (a) prior to a treatment session, (b) during a treatment session or (c) at the end or immediately after a treatment session. The disclosed system may also be configured for manual operation. For example, a care-taker or other operator performs steps comprising of (a) assessment of the measured responsiveness, (b) comparing time elapsed measurements of responsiveness, (c) determination of which eye is of lesser health, (d) determination the level of change in responsiveness over time; (e) setting the parameters of the visual stimulation to each or both eyes; or any combination thereof. The disclosed system may also be semi-automatic so to require manual steps are performed.

The method for personalized and progressive treatment for Amblyopia using VEP may include one or more of the following steps: (a) determining visual stimuli characteristics to each eye, (b) establishing a baseline measurement of a parameter representing the signal passing form the eyes to the visual cortex in respond to a visual stimuli, out of a list including Latency, Amplitude or combination thereof; (c) presenting a visual stimuli having characteristics determined by setting parameters of the presentation of a video image; (d) measuring over time said parameter representing the signal passing form the eyes to the visual cortex, and comparing to said baseline measurement outcome; (e) determining an amendment of the visual stimuli characteristics based on comparing signal passing measurements over time; and (f) altering the visual stimuli by setting new parameters of the presentation of a video image to each eye. In addition, determining an amendment or change of the visual stimuli to each eye is performed with respect to a predefined threshold. The determining an amendment of the visual stimuli may be re-performed over time.

Depending on the embodiment, the visual stimuli used for test purposes, may be the visual stimulation used for treatment purposes, and vice versa. In addition, discriminating which eye is of lesser health, is performed with respect to predefined threshold. In an embodiment of the present invention, the system is configured to detect a state of the user's eye being shut. For example if a patient fell asleep during treatment or assessment this would be detected by the system. In such case, the system incorporates at least one threshold value indicative of range of a signal transfer from eye to the brain, for determining whether or not the eye was shut. Thus, the signal is not with a threshold range definition.

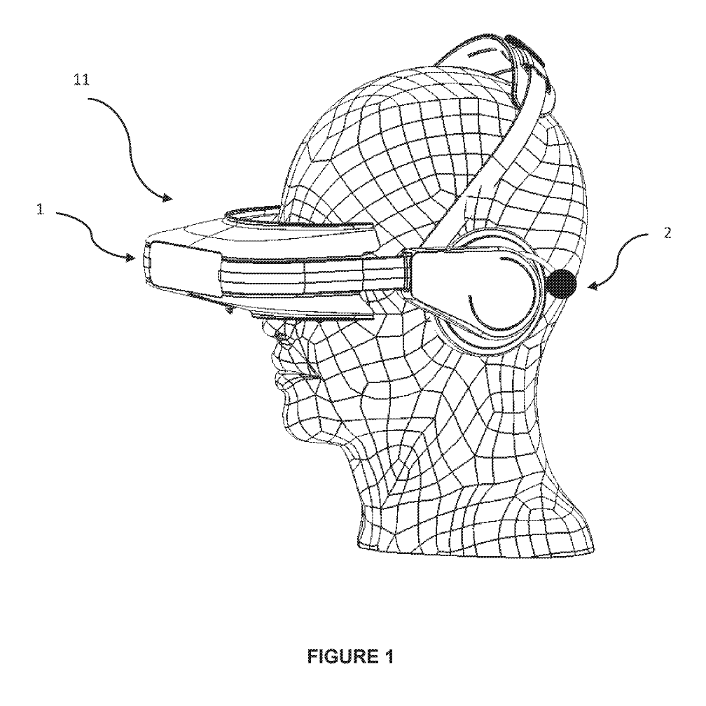

The reference is made to FIG. 1 that in exemplified way illustrates the system in one of the embodiments of the present invention. FIG. 1 shows a head mounted mechanical arrangement 11, comprising a display 1 for delivering a visual stimulation, and an electrode 2 for measuring the signal formed in responses to the visual stimulation.

The head mountable display device is configured to at least substantially isolate the user's eyes from external visual disturbances such as ambient light. In one of the embodiments the display device can fit tightly to the user's face, as goggles; in other embodiments the display device may have shades that prevent the light reaching the eyes of a user. The visual content display device can be represented by several embodiments. In one of the embodiments of the present invention the head mountable display device comprises left and right displays, each comprising one displaying area, configured to display the video content to the two eyes. In other embodiments of the present invention the head mountable visual content display device comprises a single display, by way of a non-limiting example. Such a display is of the type used in cellular phones, with left and right predefined displaying areas configured to display the video content to the two eyes. The visual content display device may also be coupled to an audio device which can be represented by a set of headphones or speakers. The headphones or speakers are configured to comprise various transducers technologies known in the art such as of electrostatic, orthodynamic, dynamic, electret, balanced armature, the Heil air motion transformer (AMT), piezoelectric film, ribbon planar magnetic, magnetostriction, plasma-ionization and electromagnetic technologies.

Depending on the embodiment a controller unit may be in communication with the video content display device and configured to alter in some embodiments the display parameters affecting image presentation on the binocular display device according to the predetermined regime. In other embodiments of the present invention the controller unit is configured to modify the display parameters affecting image presentation of at least one predefined displaying area according to the predetermined regime. In some other embodiments of the present invention the controller may comprise a graphic processing unit (GPU) configured to alter the display parameters affecting image presentation according to the predetermined regime. The display parameters affecting image presentation, depending on the embodiment, may be defined as brightness, contrast, saturation, resolution, sharpness, or any other parameter known in the art and any combination thereof. In other embodiments of the present invention the controller unit may be configured to alter the video content presentation displayed on the display device by image processing according to the predetermined regime. Moreover, the image processed video content is configured to be displayed in a dichoptic presentation such as is known in the art for presenting 3D videos, discloses, by way of non-limiting example, the group consisting of Side by Side, Frame Sequential, and Field Sequential presentation. Moreover, the controller unit may generate or trigger at least one overlay of at least one object and at least one flicker event according to the predetermined protocol. The controller unit is configured to trigger audio data coherently connected with inserting of at least one object and configured to synchronize the appearance of at least one object and the triggered audio data. The controller unit may also be in communication to the visual content source via TRS minijack, RCA jack, TOSLINK, BNC, D-subminiature, RCA jack, Mini-DIN, 3 RCA jacks, VIVO, DVI, SCART, HDMI, Display Port connector, FireWire, WiFi and Bluetooth or any type of communication link known. The data storage device is connected to the controller unit and configured to store the predetermined protocol, at least one file with at least one overlaying object and audio data. In some embodiments of the present invention the data storage device can be connected to the controller unit via PATA, IDE, EIDE, SATA, SCSI, SAS, Fiber Channel, IEEE 1394, USB, WiFi, and Bluetooth connectors. In the preferred embodiments of the present invention the data storage device is connected to the controller unit via wireless technology such as WiFi or Bluetooth. In some embodiments of the present invention the visual content source can be represented in a non-limiting way by a visual content source selected from the group consisting of a video game console, a television, a computer, a digital camera, a camcorder, a DVD, a mobile phone, a portable media player, an offline video content storage device, and a network online streaming video content or any other content source known in the art..

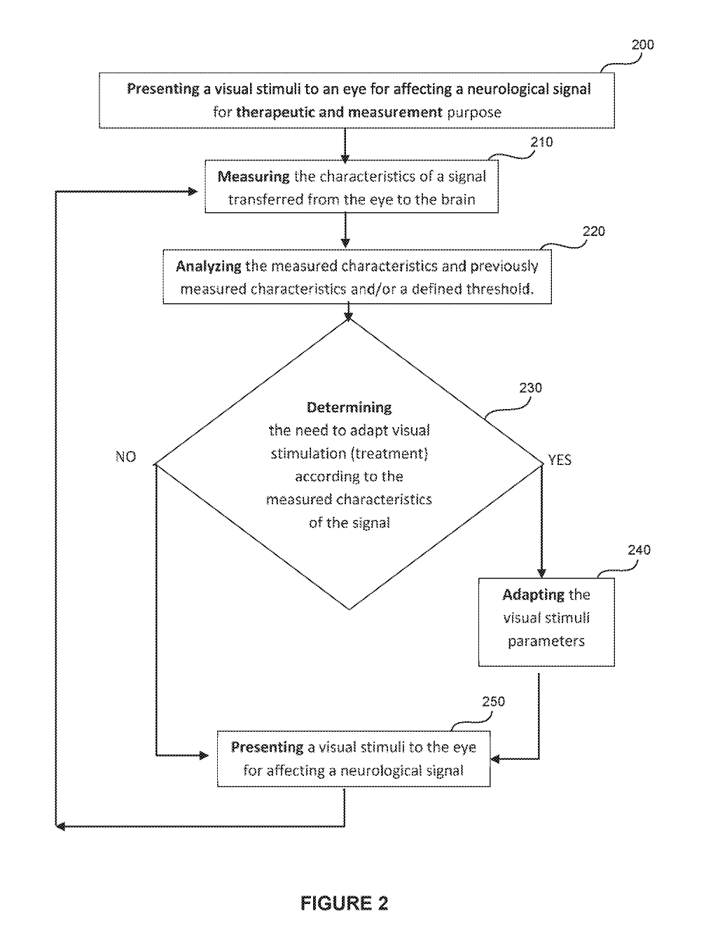

FIG. 2 depicts the steps of a method for automatic evaluation of responsiveness to a visual stimulation based treatment, and adjusting the stimulation parameters in real-time. Shown and described in FIG. 2 are steps 200-250 that depict various procedures including presenting a visual stimuli to an eye for affecting a neurological signal for therapeutic and measurement purpose.

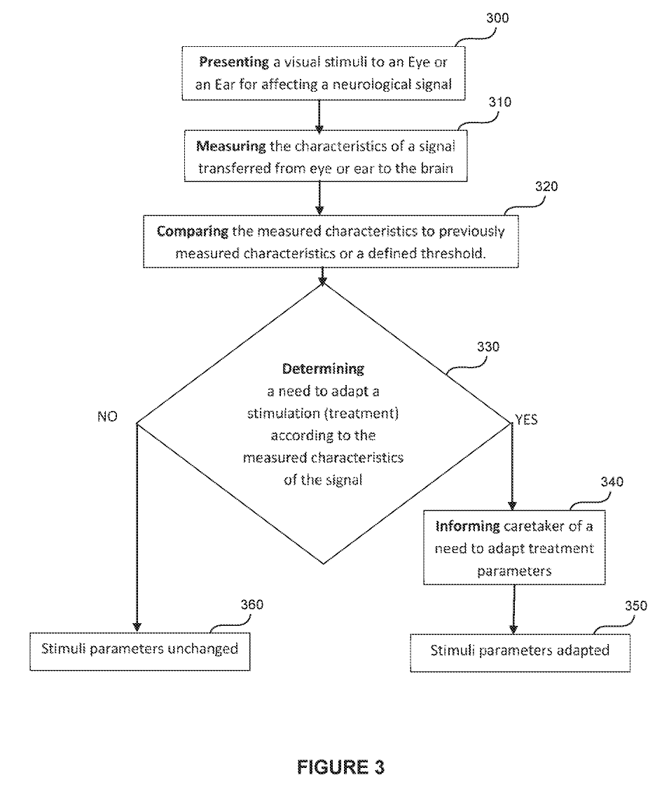

FIG. 3 depicts steps of a method for evaluation of responsiveness to a stimulation based treatment, and the same stimulation is used to test responsiveness, treat or both. The steps comprise informing a caretaker of a need to adjust parameters. Shown and described in steps 300-360 are steps that include presenting a visual stimuli to an Eye or an Ear for affecting a neurological signal.

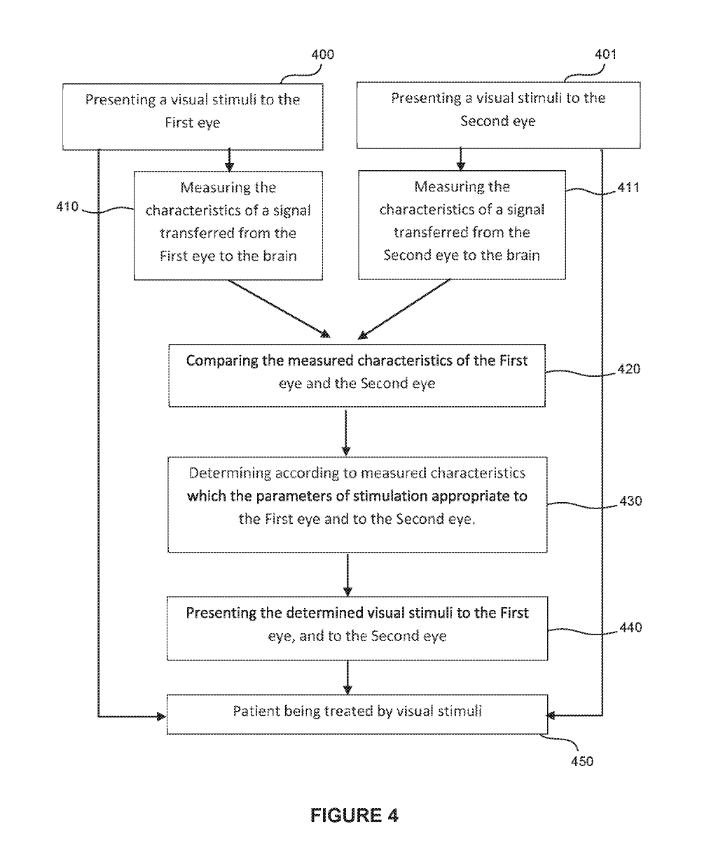

FIG. 4 depicts the steps of a method for automatic discrimination between two eyes of a person to identify the eye with lesser health and in need for treatment. The figure also depicts the same stimuli may be used either for test purposes or for treatment purpose. Steps 400-450 shown and described illustrate presenting a visual stimuli to a first eye and second eye.

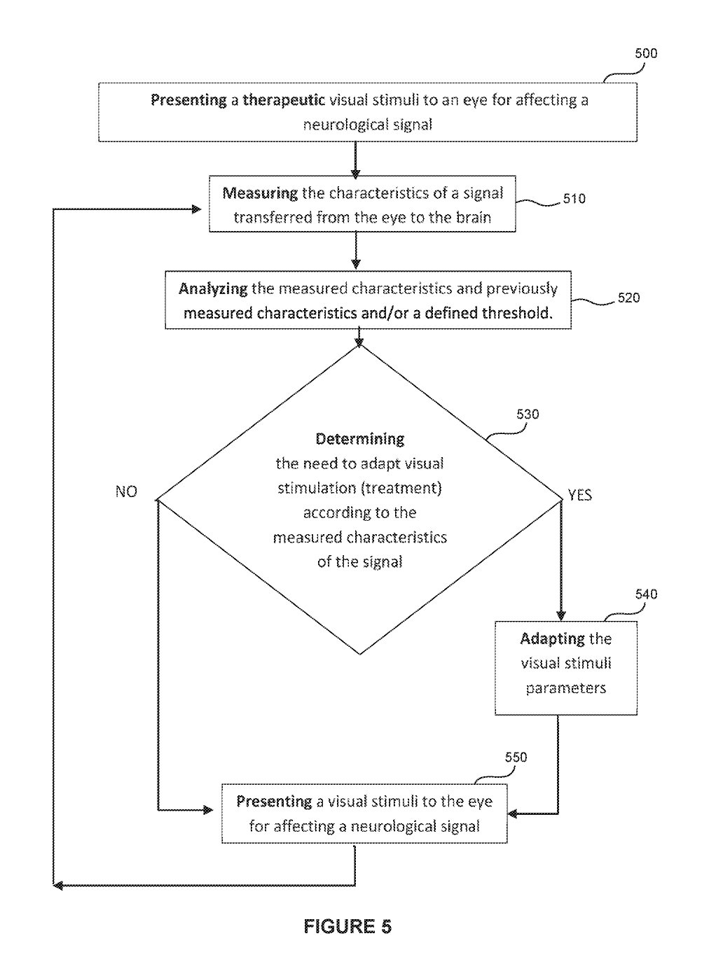

FIG.5 depicts the steps of a method for automatic evaluation of responsiveness to a visual stimulation based treatment, using therapeutic stimuli and adjusting the stimulation parameters. Presenting therapeutic visual stimuli to an eye for affecting a neurological signal are included in the steps shown in described in FIG. 5 labeled steps 500-550.

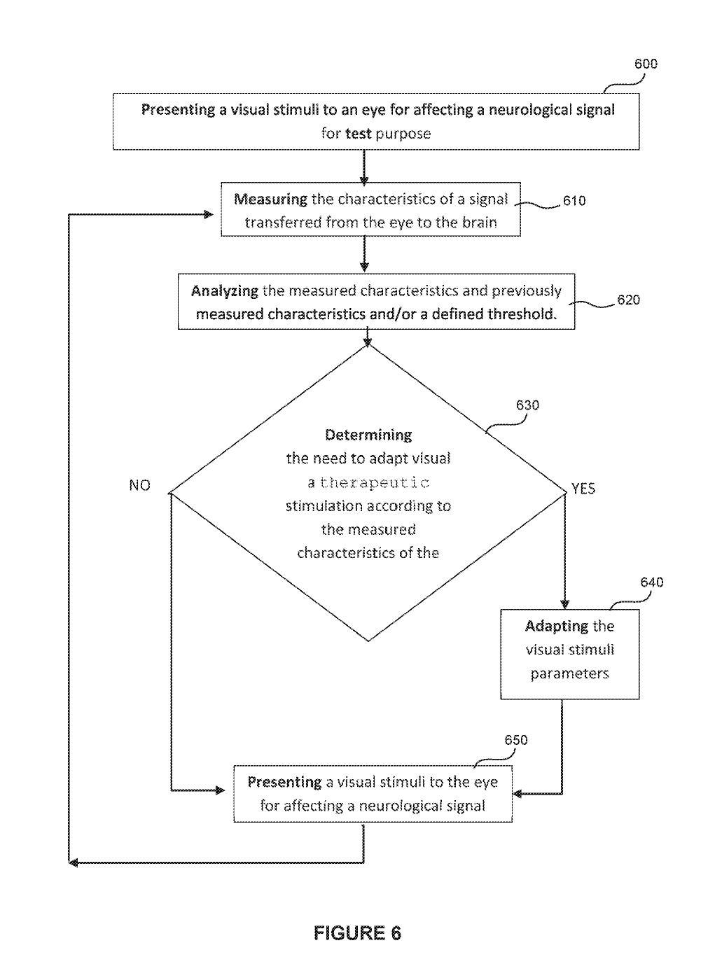

FIG.6 depicts the steps of a method for automatic evaluation of responsiveness to a visual stimulation based treatment, using special test stimuli and adjusting the stimulation parameters. Steps in FIG.6 illustrated in steps 600-650 include presenting a visual stimuli to an eye for affecting a neurological signal for a test purpose.

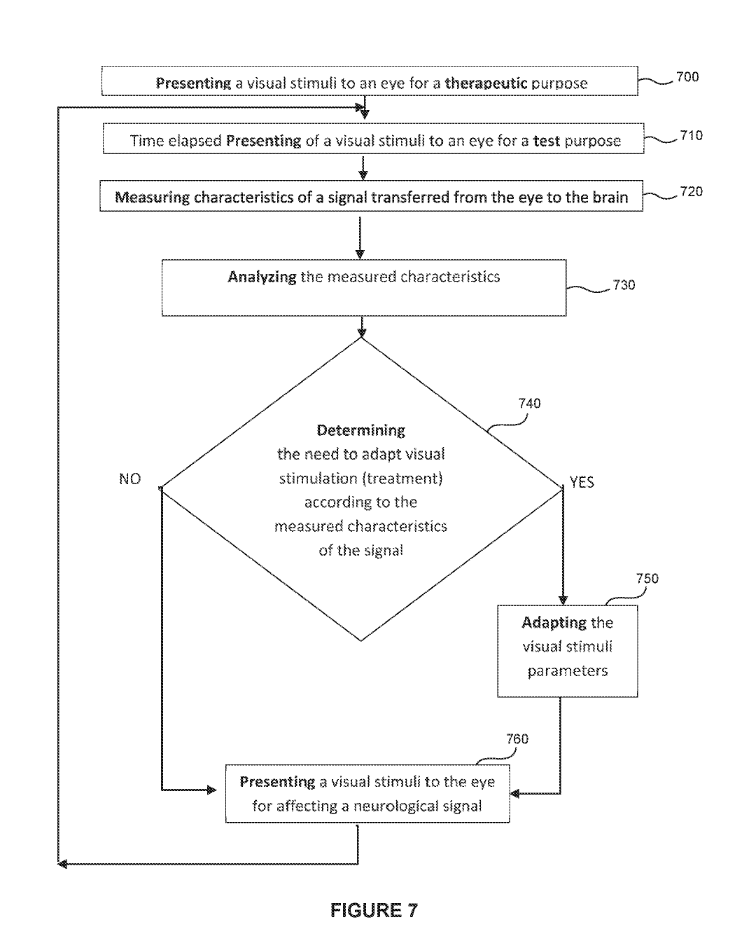

FIG.7 depicts the steps of a method for automatic evaluation of responsiveness to a visual stimulation based treatment, adjusting the stimulation parameters over time based on time elapsed re-evaluation of said responsiveness. Steps 700-760 illustrated in FIG. 7 include presenting a visual stimuli to an eye for a therapeutic purpose.

In the foregoing description, embodiments of the invention, including various embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. A system for visual stimulation of the brain by altering image parameters, in accordance to an input from an automatically measured physiological parameter, comprising:
at least one display for delivering a visual stimulation;
a controller unit coupled to the display for controlling a parameter of the visual stimulation; and
wherein the controller unit utilizes a responsiveness to a treatment to determine the parameter of the visual stimulation.

2. The system of claim 1, wherein the controller unit determines the responsiveness to the treatment based on computation of a Visual Evoked Potential (VEP) signal.

3. The system of claim 1, further comprising:
at least one electrode coupled to the controller unit, wherein the electrode is in contact with a patient's sculpt, and
a unit for sensing an electrical signal.

4. The system of claim 1, further comprising a mechanical arrangement for affixing the display and the electrode on a patient's head.

5. The system of claim 1, wherein the controller unit determines the parameter of a presented image based on computation of a Visual Evoked Potential (VEP) signal.

6. The system of claim 1, wherein the display is segmented into at least two areas, each area is visible to one eye of a user and each eye is stimulated according to the parameter adapted to a state of health of each eye.

7. The system of claim 1, wherein the stimulation further includes a video source external.

8. The system of claim 1, wherein the visual stimulation further includes a video stored on a memory component.

9. A method of treating a visual disorder, comprising measuring a physiological parameter or a state of health of a patient's eyes, prior, during, or after applying a treatment;
automatically discriminating between the state of health of the patient's eyes;
automatically measuring responsiveness over time to the treatment;
automatically determining a due change in the parameter or a stimulation of the treatment;
initiating a feedback to a patient or a caretaker;
optimizing the parameter of the treatment; and
identifying and alerting a state of awakeness.

10. A method for personalized and progressive treatment for Amblyopia using a Visual Evoked Potential (VEP) signal, comprising:
determining a visual stimuli characteristic to each one of a pair of eyes;
establishing a baseline measurement of a physiological parameter representing a signal passing from the eyes to a visual cortex in respond to a visual stimuli selected from a group consisting of latency, amplitude and any combination thereof;
presenting the visual stimuli having the characteristic determined by setting a parameter of a presentation of a video image;
measuring over time the physiological parameter representing the signal passing from the eyes to the visual cortex, and comparing to the baseline measurement outcome;
determining a change of the visual stimuli characteristic based on comparing the signal passing measurements over time; and
altering the visual stimuli by setting a new parameter of the presentation of the video image to each eye.

11. The method of claim 10, further includes determining a change of the visual stimuli to each eye is performed with respect to a predefined threshold.

12. The method of claim 11, wherein the determining the change of the visual stimuli is re-performed over time.

13. A method for evaluating and treating visual health, comprising:
presenting a visual stimulation to a patient's eye for either a therapeutic purpose or for affecting a neurological signal;
measuring a characteristic of a signal transferred from the patient's eye to the patient's brain;
analyzing the measured characteristic of the signal;
determining a need to adapt the visual stimulation according to the measured characteristic of the signal; and
adapting a parameter of the visual stimulation or presenting the visual stimulation to the patient's eye for affecting the neurological signal;

14. The method of claim 13, further includes returning to measuring the characteristic of the signal transferred from the patient's eye to the patient's brain.

15. The method of claim 13, further includes presenting via time elapsed the visual stimulation to the patient's eye.

16. The method of claim 15, wherein the presenting via time elapsed the visual stimulation to the patient's eye is done for a test purpose rather than a treatment purpose.

17. The method of claim 15,wherein the presenting via time elapsed is done before the step of measuring the characteristics of the signal transferred from the patient's eye to the patient's brain.

18. The method of claim 17, wherein after the step of adapting the parameter of the visual stimulation or the step of presenting the visual stimulation to the patient's eye for affecting the neurological signal further includes returning to presenting via time elapsed the visual stimulation to the patient's eye is done.

19. The method of claim 13, wherein if the step of determining the need to adapt visual stimulation according the measured characteristics of the signal is positive, then the step of adapting visual stimulation is sequentially done next, and if negative then the step of presenting of the visual stimulation to the eye for affecting the neurological signal is sequentially done next instead of the determining step.

20. The method of claim 13, wherein the step of analyzing the measured characteristics further includes a previously measured characteristic, a predefined threshold, and any combination thereof.
